(19) United States
(12) Patent Application Publication
(10) Pub. No.: US 2022/0233784 A1
(43) Pub. Date: Jul. 28, 2022

(54) DRUG DELIVERY DEVICE

(71) Applicant: Sanofi, Paris (FR)

(72) Inventors: Michael Jugl, Frankfurt am Main (DE); Stefan Blancke, Frankfurt am Main (DE)

(21) Appl. No.: 17/612,591

(22) PCT Filed: May 27, 2020
(86) PCT No.: PCT/EP2020/064707

§ 371 Date: Nov. 19, 2021

(30) Foreign Application Priority Data
May 29, 2019 (EP) 19305680.1

(51) Int. Cl. A61M 5/32; A61P 3/10; A61M 5/24; A61M 5/315; C07K 14/62

(52) U.S. Cl. CPC A61M 5/329; A61P 3/10; A61M 5/2448; A61M 2207/10; C07K 14/62; A61M 2005/2407; A61M 2205/0238; A61M 5/3155

(57) ABSTRACT

The present disclosure relates to a drug delivery device. The drug delivery device comprises a drug container containing a drug liquid, wherein the drug liquid includes insulin glargine dissolved therein, and a hollow needle defining an elongated interior space through which the drug liquid is dispensable from the drug container, wherein the needle is at least partially made of a steel resistant to chloride, wherein the elongated interior space is in fluid communication with the drug container in a storage or pre-delivery condition, for example within a timeframe of at least 1 hour in absence of significant exchange of drug liquid inside the interior space.

### CROSS REFERENCE TO RELATED APPLICATIONS

The present application is the national stage entry of International Patent Application No. PCT/EP2020/064707, filed on May 27, 2020, and claims priority to Application No. EP 19305680.1, filed on May 29, 2019, the disclosures of which are incorporated herein by reference.

### TECHNICAL FIELD

The present disclosure relates to a drug delivery device. In particular, the present disclosure relates to a method for manufacturing a drug delivery device and to a method for administration a dose of a drug using a drug delivery device.

### BACKGROUND

Many liquids, such as medicaments, have to be injected into the body. This applies in particular to medicaments which are deactivated or have their efficiency remarkably decreased by oral administration, e.g., proteins (such as insulin, growth hormones, interferons), carbohydrates (e.g., heparin), antibodies, and the majority of vaccines. Such medicaments are predominantly injected by means of delivery devices such as syringes, medicament pens, or medicament pumps.

The user of such syringes, medicament pens, or medicament pumps can range from healthcare professionals to the medicament-recipient themselves, the latter ranging from children or elderly persons. The medicinal injections may include repetitive or multiple injections of a particular dose (e.g., a vaccine in multi-dosage regimen) to a single injection of a single dose (e.g., a vaccine or in an emergency hydrocortisone).

For this purpose, there are several types of medication delivery devices known such as pen type delivery devices. These delivery devices have in common that they are configured deliver the drug liquid through a hollow needle made of steel.

Despite the advantages provided by these delivery devices, there are still some drawbacks. Usually the needle is made of a stainless steel such as material number 1.4301 according to EN 10027. However, not any stainless steel is resistant to chloride. If such a drug delivery device is used for administration of a drug comprising insulin glargine dissolved therein, the drug tends to clog, particularly in or near such a needle. As such, the drug cannot be properly dispensed through the needle. Such clogging is likely caused by a reaction of the insulin glargine with the steel of the needle when they contact one another, as insulin glargine is formulated at an acidic pH 4, where it is completely water-soluble, and comprises chloride. Such clogging is undesirable because it can prevent patients from receiving full doses of the drug. Thus, prevention of needle clogging can be crucial for allowing provision of the drug.

### SUMMARY

It is an aspect of the present disclosure to minimize or eliminate clogging of a needle of a drug delivery device used in combination with a drug liquid comprising insulin glargine dissolved therein and thereby allowing for a proper provision of the drug liquid via the drug delivery device.

In a first aspect of a general concept, there is provided a drug delivery device which comprises a drug container containing a drug liquid, wherein the drug liquid comprises insulin glargine dissolved therein, and a hollow needle defining an elongated interior space through which the drug liquid is dispensable from the drug container, wherein the needle is at least partially made of a steel resistant to chloride. The elongated interior space is flow connected or in fluid communication to the drug container in a storage or pre-delivery condition, for example, within a timeframe of at least 1 hour in absence of significant exchange of drug liquid inside the interior space. Thus, even though a fluidic contact of the drug liquid comprising the insulin glargine and the material of the needle at the interior space may be present, clogging is prevented as this type of steel is resistant to a solution comprising insulin glargine. Particularly, this type of steel is non-corrosive when contacted by a solution comprising insulin glargine.

According to a further embodiment, the steel comprises molybdenum. Thus, the steel is well resistant to solutions comprising chloride. In this respect, it has to be noted that such types of steel are more expensive than a steel not containing molybdenum such that it is usually refrained from using a steel containing molybdenum for costs reasons.

According to a further embodiment, the steel comprises molybdenum in 1.5 to 7.0% by mass, 1.75 to 6.5% by mass, or 2.0 to 6.0% by mass. Thus, the steel is even more resistant to solutions comprising chloride.

According to a further embodiment, the steel is at least one steel selected from the group consisting of: material number 1.4104, 1.4113, 1.4125, 1.4401, 1.4404, 1.4406, 1.4429, 1.4435, 1.4436, 1.4438, 1.4439, 1.4449, 1.4460, 1.4462, 1.4521, 1.4529, 1.4539, 1.4565, 1.4571 according to EN 10027. These types of steel are most suitable for use with drugs comprising insulin glargine as they comprise molybdenum which increases the resistance to chloride.

According to a further embodiment, the drug delivery device further comprises a body comprising a receptacle configured to receive the drug container, wherein the needle is removably connectable to the body so as to be in fluid communication with the drug container. Thus, the needle may be replaced after use by a new one without the need to dispose the whole drug delivery device. Further, the drug container may be replaced after use by a new one without the need to dispose the whole drug delivery device.

According to a further embodiment, the drug delivery device further comprises a body comprising a receptacle configured to receive the drug container, wherein the needle is permanently connected to the body so as to be in fluid communication with the drug container. Thus, the drug container may be replaced after use by a new one without the need to dispose the whole drug delivery device.

According to a further embodiment, the drug delivery device further comprises a dose setting member connected to the body and configured to set a dose of the drug liquid. Thus, the dose to be dispensed may be adjusted by the user of the drug delivery device.

According to a further embodiment, the drug delivery device further comprises a dose button operable for dispensing a dose of the drug liquid set by the dose setting member. The provision of a dose button improves the operability for the user of the drug delivery devise as the user only needs to push the dose button for dispensing the drug liquid.

According to a further embodiment, the body comprises a distal end to which the needle is connected or connectable and a proximal end to which the dose setting member is connected, wherein the distal end and the proximal end are spaced apart from one another in the direction of an axis. Thus, by operating the dose setting member in an axial direction, the dose may be set or the drug liquid may be dispensed.

According to a further embodiment, the needle is completely made of the steel resistant to chloride.

This further improves the resistance of the needle to chloride and facilitates its manufacturing process.

Alternatively or in addition, the needle surface defining the elongated interior space has a coating facing towards the elongated interior space, wherein the coating is made of the steel resistant to chloride. With this embodiment, the outer portion of the hollow needle facing away from the inner channel or interior space may be made of any material such as a steel having no or not fully satisfying resistance to chloride.

According to a further embodiment, the drug container and the hollow needle may be components of a pre-filled syringe. Thus, the drug container and the hollow needle are pre-assembled such that the contact of the liquid drug and the material of the needle at the surface defining the interior space may be present for a rather long time. Despite this long time contact, clogging may be reliably prevented.

According to a further embodiment, the drug delivery device is configured to dispense a plurality of doses of the drug liquid from the drug container. Thus, the drug delivery device may be used to dispense a plurality of single doses from the drug container. Thereby, the drug container does not need to be replaced after each single dispensing operation.

According to a further embodiment, the drug delivery device is a pen type delivery device. With such a type of drug delivery device, most users a familiar such that the handling thereof is rather easy.

According to a further embodiment, the needle is a small gauge needle in a range of 22 gauge to 32 gauge and preferably in a range of 24 gauge to 31 gauge. Thus, most common needle types may be used with the drug delivery device.

According to a further embodiment, the drug comprises 200 to 1000 U/ml [equimolar to 200 to 1000 IU/ml human insulin] of insulin glargine. Thus, a sufficient amount of insulin glargine may be used with the drug delivery device.

According to a further embodiment, the drug liquid comprises a buffer comprising hydrochloric acid in an amount such that the drug liquid comprises a pH value of, for example, 1 to 6.8, 3.5 to 6.8, or 3.5 to 4.5.

According to a further embodiment, the buffer comprises 0.1 M hydrochloric acid.

In a second aspect of a general concept, there is provided a method for manufacturing a drug delivery device of any preceding embodiment comprising:
providing a drug container containing a drug liquid, wherein the drug liquid comprises insulin glargine dissolved therein, and
making a hollow needle, which defines an elongated interior space through which the drug liquid is dispensable from the drug container, at least partially of a steel resistant to chloride. The elongated interior space is flow connected or in fluid communication to the drug container in a storage or pre-delivery condition, in particular within a timeframe of at least 1 hour in absence of significant exchange of drug liquid inside the interior space.

In a third aspect of a general concept, there is provided a method for administration a dose of a drug liquid, wherein the drug liquid comprises insulin glargine dissolved therein, using a drug delivery of any preceding embodiment, comprising:
providing a drug container containing a drug liquid, wherein the drug liquid comprises insulin glargine dissolved therein, and,
providing a hollow needle, which defines an elongated interior space, wherein the needle is at least partially made of a steel resistant to chloride, and
dispensing the dose of the drug liquid through the interior space of the needle from the drug container.

Summarizing the findings of the present disclosure, the following embodiments are disclosed:

Embodiment 1: A drug delivery device comprising:
a drug container containing a drug liquid, wherein the drug liquid comprises insulin glargine dissolved therein, and
a hollow needle defining an elongated interior space through which the drug liquid is dispensable from the drug container, wherein the needle is at least partially made of a steel resistant to chloride,
wherein the elongated interior space is in fluid communication with the drug container in a storage or pre-delivery condition, in particular within a timeframe of at least 1 hour in absence of significant exchange of drug liquid inside the interior space.

Embodiment 2: The drug delivery device of any preceding embodiment, wherein the steel comprises molybdenum.

Embodiment 3: The drug delivery device of embodiment 2, wherein the steel comprises molybdenum in 1.5 to 7.0% by mass, e.g., 1.75 to 6.5% by mass or 2.0 to 6.0% by mass.

Embodiment 4: The drug delivery device of any preceding embodiment, wherein the steel is at least one steel selected from the group consisting of: material number 1.4104, 1.4113, 1.4125, 1.4401, 1.4404, 1.4406, 1.4429, 1.4435, 1.4436, 1.4438, 1.4439, 1.4449, 1.4460, 1.4462, 1.4521, 1.4529, 1.4539, 1.4565, 1.4571 according to EN 10027.

Embodiment 5: The drug delivery device of any preceding embodiment, further comprising a body comprising a receptacle configured to receive the drug container, wherein the needle is removably connectable to the body so as to be in fluid communication with the drug container.

Embodiment 6: The drug delivery device of any one of embodiments 1 to 4, further comprising a body comprising a receptacle configured to receive the drug container, wherein the needle is permanently connected to the body so as to be in fluid communication with the drug container.

Embodiment 7: The drug delivery device of embodiment 5 or 6, further comprising a dose setting member connected to the body and configured to set a dose of the drug liquid.

Embodiment 8: The drug delivery device of embodiment 7, further comprising a dose button operable for dispensing a dose of the drug liquid set by the dose setting member.

Embodiment 9: The drug delivery device of any one of embodiments 6 to 8, wherein the body comprises a distal end to which the needle is connected or connectable and a proximal end to which the dose setting member is connected, wherein the distal end and the proximal end are spaced apart from one another in the direction of an axis.

Embodiment 10: The drug delivery device of any preceding embodiment, wherein the needle is completely made of the steel resistant to chloride.

Embodiment 11: The drug delivery device of any one of embodiments 1 to 9, wherein the needle surface defining the elongated interior space has a coating facing towards the elongated interior space, wherein the coating is made of the steel resistant to chloride.

Embodiment 12: The drug delivery device of any preceding embodiment, wherein the drug container and the hollow needle are components of a pre-filled syringe.

Embodiment 13: The drug delivery device of any preceding embodiment, wherein the drug delivery device is configured to dispense a plurality of doses of the drug liquid from the drug container.

Embodiment 14: The drug delivery device of any preceding embodiment, wherein the drug delivery device is a pen type delivery device.

Embodiment 15: The drug delivery device of any preceding embodiment, wherein the needle is a small gauge needle in a range of 22 gauge to 32 gauge or in a range of 24 gauge to 31 gauge.

Embodiment 16: The drug delivery device of any preceding embodiment, wherein the drug comprises 200 to 1000 U/ml [equimolar to 200 to 1000 IU/ml human insulin] of insulin glargine.

Embodiment 17: The drug delivery device of any preceding embodiment, wherein the drug liquid comprises a buffer comprising hydrochloric acid in an amount such that the drug liquid comprises a pH value of 1 to 6.8, 3.5 to 6.8, or 3.5 to 4.5.

Embodiment 18: The drug delivery device of the preceding embodiment, wherein the buffer comprises 0.1 M hydrochloric acid.

Embodiment 19: A method for manufacturing a drug delivery device of any preceding embodiment comprising:
providing a drug container containing a drug liquid, wherein the drug liquid comprises insulin glargine dissolved therein, and
making a hollow needle, which defines an elongated interior space through which the drug liquid is dispensable from the drug container, at least partially of a steel resistant to chloride, wherein the elongated interior space is in fluid communication with the drug container in a storage or pre-delivery condition, in particular within a timeframe of at least 1 hour in absence of significant exchange of drug liquid inside the interior space.

It shall be understood that the drug delivery device of embodiment 1 and the method of embodiment 15 have similar and/or identical preferred embodiments as defined in the dependent embodiments.

It shall be understood that a preferred embodiment of the disclosure can also be any combination of the dependent claims or above embodiments with the respective independent claim.

These and other aspects of the disclosure will be apparent from and elucidated with reference to the embodiments described hereinafter.

### BRIEF DESCRIPTION OF THE FIGURES

In the following drawing:

FIG. 1 shows schematically and exemplarily an embodiment of a drug delivery device.

### DETAILED DESCRIPTION

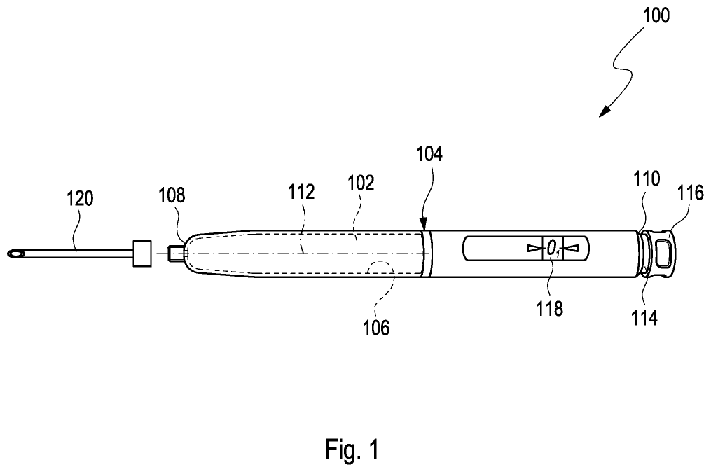

FIG. 1 shows schematically and exemplarily an embodiment of a drug delivery device 100.

An example application of the disclosure is in administration of a drug liquid. It is specifically designed for use in administration of a drug liquid comprising insulin glargine, but basically it can also be used for administration of a drug liquid comprising pharmaceutical active ingredients other than insulin glargine.

Other variations to the disclosed embodiments can be understood and effected by those skilled in the art in practicing the claimed disclosure, from a study of the drawings, the disclosure, and the appended claims.

As used herein, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Determinations like measuring, et cetera performed by one or several units or devices can be performed by any other number of units or devices. For example, detecting can be performed by a single unit or by any other number of different units. The determinations and/or the control of the system for use in accordance with the above described method for manufacturing the drug delivery device or administration of a dose can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems. The term “computer program” may also refer to embedded software.

Any reference signs in the claims should not be construed as limiting the scope.

The terms “drug liquid” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

The drug liquid or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug liquid for at least one day (e.g., 1 to at least 30 days). In some instances, the chamber may be designed to store a drug liquid for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism, etc. Further examples of disorders are acute coronary syndrome (ACS), angina, myocardial infarction, cancer, macular degeneration, inflammation, hay fever, atherosclerosis, and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

An aspect of the present disclosure relates to a drug liquid comprising insulin glargine. Insulin glargine is 31^{B}-32^{B}-Di-Arg human insulin, an analogue of human insulin, with further substitution of asparagine in position A21 by glycine. Further details regarding the formulation of insulin glargine can be taken from WO 2011/144673 A2, the contents thereof regarding the details of the formulation of insulin glargine are explicitly incorporated herein by reference.

Lantus® is an insulin product containing insulin glargine providing 24 hour basal insulin supply after single dose subcutaneous injection.

The glucodynamic effect of Lantus® is distinguished from other currently marketed insulin products by virtue of a delayed and predictable absorption of insulin glargine from the subcutaneous injection site resulting in a smooth, 24 hour time-concentration and action profile without a definite peak. Lantus® was developed to meet the medical need for a long-acting insulin product that can be administered as a single daily injection to yield normal or near-normal blood glucose control with a basal insulin profile that is as smooth as possible over a 24-hour period. Such a preparation provides good control of blood glucose all day, while minimizing the tendency to produce hypoglycemia seen with other insulin preparations with a more definite “peak” effect. A considerable number of patients, e.g., those with increased insulin resistance due to obesity, use large doses to control blood glucose. For example, a dose of 100 U requires injection of 1 mL Lantus® U100, which may confer some discomfort; each mL Lantus® U100 contains 100 U (3.6378 mg) insulin glargine. To reduce the volume of injection, a formulation containing 300 U insulin glargine per mL has been developed. Although the disclosure is not limited to an insulin glargine U 300 formulation, the clinical studies described herein were performed with an insulin glargine U 300 formulation; each mL insulin glargine U300 contains 300 U (10.9134 mg) insulin glargine. This formulation would allow patients to inject the same number of units of insulin glargine at one third the volume of injection.

Both insulin glargine formulations, U100 and U300, were expected to provide the same insulin exposure and the same effectiveness, i.e., time profiles.

As used herein, the term “resistant to chloride” refers to the material characteristics of a solid material not to or substantially not to form a chemical compound with chloride or to be dissolved by chloride, for example, due to corrosion, even at a high temperature. Needless to say, as the present disclosure relates to the medical or pharmaceutical field, the resistance to solutions comprising chloride applies to those solutions, mixtures and concentrations thereof which are usually used in this technical field. The characteristics of being resistant to solutions comprising chloride may be determined by exposition of the needle to the respective solution such as a solution comprising HCl or insulin glargine for a predetermined time and subsequently detecting the surface roughness of the needle. Alternatively, the pitting corrosion of the material of the needle may be detected such as in potentiometric manner. Thereby, the pitting corrosion potential or pitting resistance equivalent number may be determined indicating the material resistance to corrosion.

The drug delivery device 100 shown in FIG. 1 is a pen type delivery device as will be described in further detail below. The drug delivery device 100 comprises a drug container 102 containing a drug liquid. The drug liquid comprises insulin glargine dissolved therein. Particularly, the drug liquid comprises 200 to 1000 U/ml [equimolar to 200 to 1000 IU/ml human insulin] of insulin glargine. The drug container 102 may be a cartridge or the like. The drug delivery device 100 is a pen type delivery device 100 as will be described in further detail below. The drug delivery device 100 is configured to dispense a plurality of doses of the drug liquid from the drug container 102.

The drug delivery device 100 further comprises a body 104 comprising a receptacle 106 configured to receive the drug container 102. For example, the receptacle is designed as a cartridge holder. The body 104 comprises a distal end 108 and a proximal end 110. The distal end 108 and the proximal end 110 are spaced apart from one another in the direction of an axis 112. The drug container 102 can be loaded into the receptacle 106 from a proximal end section thereof.

The drug delivery device 100 further comprises a dose setting member 114 connected to the body 104 and configured to set a dose of the drug liquid. More particularly, the dose setting member 114 is connected to the proximal end 110 of the body 104. The drug delivery device 100 further comprises a dose button 116 operable for dispensing a dose of the drug liquid set by the dose setting member 114. The dose button 116 is located at a proximal end section of the dose setting member 114. The drug delivery device 100 comprises a piston rod (not shown in detail). The piston rod is configured to transfer movement through the body 104 for expelling a dose of drug liquid from the cartridge. The piston rod is moveable between an initial position with respect to the body 104 and an end position with respect to the body 104. The initial position may be the position of the piston rod when the drug delivery device 100 is supplied from the manufacturer. Moreover, the initial position may be the position of the piston rod after a reset operation was performed. The initial position may be the most proximal position of the piston rod. The end position may be the position of the piston rod after the complete amount of the drug liquid was dispensed from the cartridge. The end position may be the most distal position of the piston rod. During operation of the drug delivery device 100, in particular for dispensing a dose of the drug liquid, the piston rod is moved towards the end position.

The piston rod has a distal end, which is arranged nearest to the dispensing end of the drug delivery device 100. The distal end section of the piston rod comprises a bearing member. The bearing member is arranged between the bung and the piston rod. The bearing member is configured to reduce damages that may be caused by friction. The bearing member may be part of the piston rod. The bearing member may be connected to the piston rod. Alternatively, the bearing member and the piston rod may be integrally formed. The bearing member and the bung are in mechanical contact, for example in abutment, throughout the operation of the device. The bearing member and the bung are in mechanical contact as long as the cartridge or a replacement cartridge is loaded within the device. In other words, the bearing member and the bung are in mechanical contact as long as the receptacle 106 is at least partly connected to the body 104.

The piston rod is configured as a lead screw. The piston rod comprises two threaded sections. The threaded sections have opposite senses of rotation. A first threaded section is located at a distal part of the piston rod and a threaded section is located at a proximal part of the piston rod. The piston rod and, e.g., the first threaded section, is in threaded engagement with a guiding member (not shown in detail), e.g., a guide nut. The guiding member comprises a centered hole. Within the centered hole, a screw thread is designed. The screw thread is used for being coupled to the piston rod in order to urge the piston rod in a predetermined helical movement through the body 104 and towards the end position. The piston rod is axially and rotationally moveable towards the end position due to mechanical cooperation with the guiding member. Furthermore, the piston rod and, e.g., the second threaded section is in threaded engagement with a drive member (not shown in detail). The drive member exerts a force onto the piston rod to cause a movement of the piston rod for delivering a dose of the drug liquid when a user pushes onto the dose button 116. A dose set by means of the dose setting member 114 is visible through a dose window 118. For example, the number units of the drug liquid set by the user is visible through the dose window 118.

The drug delivery device 100 further comprises a hollow needle 120 which defines an elongated interior space through which the drug liquid is dispensable from the drug container 102. The needle 120 is connected or connectable to the distal end 108 of the body 104. With the exemplary embodiment shown in FIG. 1, the needle 120 is removably connectable to the body 104 so as to be in fluid communication with the drug container 102. For example, the needle 120 may be threaded to the body 104. Alternatively, the needle 120 may be permanently connected to the body 104 so as to be in fluid communication with the drug container 102. The needle 120 is a small gauge needle in a range of 22 gauge to 32 gauge or in a range of 24 gauge to 31 gauge, such as 26 gauge. The elongated interior space is in fluid communication with the drug container 102 in a storage or pre-delivery condition, for example within a timeframe of at least 1 hour in absence of significant exchange of drug liquid inside the interior space.

The needle 120 is at least partially made of a steel resistant to chloride. For example, the needle 120 is made of this steel only at its inner portion facing the inner channel through the needle 102. As such, the surface of the needle 120 defining the interior space may comprise a coating facing towards the interior space thereof, which coating is made of the steel resistant to chloride. It is explicitly stated that the needle 120 may be completely made of the steel resistant to chloride. For this purpose, the steel comprises molybdenum. For example, the steel comprises molybdenum in 1.5 to 7.0% by mass, e.g., 1.75 to 6.5% by mass, 2.0 to 6.0% by mass, or 3.0% by mass. The steel is at least one steel selected from the group consisting of: material number 1.4104, 1.4113, 1.4125, 1.4401, 1.4404, 1.4406, 1.4429, 1.4435, 1.4436, 1.4438, 1.4439, 1.4449, 1.4460, 1.4462, 1.4521, 1.4529, 1.4539, 1.4565, 1.4571 according to EN 10027 such as 1.4539 according to EN 10027.

A method for manufacturing the drug delivery device 100 comprises providing the drug container 102 containing a drug liquid, wherein the drug liquid comprises insulin glargine dissolved therein. For example, the drug container 102 may be provided as a cartridge comprising a plurality of doses of insulin glargine. Further, the hollow needle 120, which defines an elongated interior space through which the drug liquid is dispensable from the drug container 102, may be manufactured by any method know to the skilled person, wherein the needle 120 is made at least partially of a steel resistant to chloride as described above such as 1.4539 according to EN 10027. Further, the drug container 102 may be loaded into the receptacle 106 of the body 104. Furthermore, the needle 120 may be designed so as to be removably or permanently connectable to the body 104.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

### LIST OF REFERENCE NUMBERS

100 drug delivery device
102 drug container
104 body
106 receptacle
108 distal end
110 proximal end
112 axis
114 dose setting member
116 dose button
120 hollow needle

1-15. (canceled)

16. A drug delivery device comprising:
a drug container containing a drug liquid, wherein the drug liquid comprises insulin glargine dissolved therein, and
a hollow needle defining an elongated interior space through which the drug liquid is dispensable from the drug container, wherein the needle is at least partially made of a steel resistant to solutions comprising chloride,
wherein the elongated interior space is in fluid communication with the drug container in a storage or pre-delivery condition.

17. The drug delivery device of claim 16, wherein the storage or pre-delivery condition is within a timeframe of at least 1 hour in absence of significant exchange of drug liquid inside the interior space.

18. The drug delivery device of claim 16, wherein the steel comprises molybdenum.

19. The drug delivery device of claim 18, wherein the steel comprises molybdenum in 1.5 to 7.0% by mass.

20. The drug delivery device of claim 18, wherein the steel comprises molybdenum in 1.75 to 6.5% by mass.

21. The drug delivery device of claim 18, wherein the steel comprises molybdenum in 2.0 to 6.0% by mass.

22. The drug delivery device of claim 16, wherein the steel is at least one steel selected from the group consisting of: material number 1.4104, 1.4113, 1.4125, 1.4401, 1.4404, 1.4406, 1.4429, 1.4435, 1.4436, 1.4438, 1.4439, 1.4449, 1.4460, 1.4462, 1.4521, 1.4529, 1.4539, 1.4565, 1.4571 according to EN 10027.

23. The drug delivery device of claim 16, further comprising:
a body comprising a receptacle configured to receive the drug container, wherein the needle is removably connectable to the body so as to be in fluid communication with the drug container or wherein the needle is permanently connected to the body so as to be in fluid communication with the drug container;
a dose setting member connected to the body and configured to set a dose of the drug; and
a dose button operable for dispensing the dose of the drug set by the dose setting member;
wherein the body comprises a distal end to which the needle is connected or connectable and a proximal end to which the dose setting member is connected, wherein the distal end and the proximal end are spaced apart from one another in the direction of an axis.

24. The drug delivery device of claim 16, wherein the needle consists essentially of the steel resistant to chloride.

25. The drug delivery device of claim 16, wherein a needle surface defining the elongated interior space has a coating facing towards the elongated interior space, wherein the coating is made of the steel resistant to chloride.

26. The drug delivery device of claim 16, wherein the drug container and the hollow needle are components of a pre-filled syringe.

27. The drug delivery device of claim 16, wherein the drug delivery device is configured to dispense a plurality of doses of the drug liquid from the drug container.

28. The drug delivery device of claim 16, wherein the drug delivery device is a pen type delivery device.

29. The drug delivery device of claim 16, wherein the needle is a small gauge needle in a range of 22 gauge to 32 gauge.

30. The drug delivery device of claim 16, wherein the needle is a small gauge needle in a range of 24 gauge to 31 gauge.

31. The drug delivery device of claim 16, wherein the drug liquid comprises a molar equivalent of 200 to 1000 IU/ml human insulin.

32. The drug delivery device of claim 31, wherein the drug liquid comprises 200 to 1000 U/ml of insulin glargine.

33. The drug delivery device of claim 16, wherein the drug liquid comprises a buffer comprising hydrochloric acid in an amount such that the drug liquid comprises a pH value of 1 to 6.8.

34. The drug delivery device of claim 33, wherein the buffer comprises 0.1 M hydrochloric acid.

35. A method for manufacturing a drug delivery device comprising a drug container containing a drug liquid, wherein the drug liquid comprises insulin glargine dissolved therein, and a hollow needle defining an elongated interior space through which the drug liquid is dispensable from the drug container, wherein the needle is at least partially made of a steel resistant to solutions comprising chloride, the method comprising:
providing the drug container containing the drug liquid comprising insulin glargine dissolved therein, and
making the hollow needle, which defines the elongated interior space through which the drug liquid is dispensable from the drug container, at least partially of the steel resistant to solutions comprising chloride, wherein the elongated interior space is in fluid communication with the drug container in a storage or pre-delivery condition within a timeframe of at least 1 hour in absence of significant exchange of drug liquid inside the interior space.
